(19) United States
(12) Patent Application Publication
(10) Pub. No.: US 2024/0307204 A1
(43) Pub. Date: Sep. 19, 2024

(54) METHOD OF AUGMENTING WEIGHT LOSS BY PYLORUS-SPARING GASTRIC MYOTOMY

(71) Applicant: EnVision Endoscopy, Inc., Waltham, MA (US)

(72) Inventor: Christopher C. Thompson, Needham, MA (US)

(21) Appl. No.: 18/676,706

(22) Filed: May 29, 2024

Related U.S. Application Data
(63) Continuation of application No. PCT/US22/51675, filed on Dec. 2, 2022.
(60) Provisional application No. 63/285,371, filed on Dec. 2, 2021.

(51) Int. Cl. A61F 5/00

(52) U.S. Cl. CPC A61F 5/0083

(57) ABSTRACT

The present invention provides methods for augmenting weight loss through metabolic surgery. More specifically, the method of the present invention includes creating a submucosal tunnel in the gastric body of a patient extending from the proximal gastric antrum to the pre-pyloric area. One or more myotomies are created in the submucosal tunnel extending from the pre-pyloric area to just distal of the opening of the submucosal tunnel. The submucosal tunnel is closed, and a running suture is performed along the greater curvature of the stomach. Optionally, an endoscopic gastroplasty may then be performed. The method of the present invention alters gastric physiology, slows gastric emptying, and augments patient weight loss.

### CROSS REFERENCE TO RELATED APPLICATIONS

This application is a continuation of International Patent Application No. PCT/US2022/051675, entitled METHOD OF AUGMENTING WEIGHT LOSS BY PYLORUS-SPARING GASTRIC MYOTOMY, which claims the benefit of U.S. Provisional Patent Application No. 63/285,371 entitled METHOD OF AUGMENTING WEIGHT LOSS BY PYLORUS-SPARING GASTRIC MYOTOMY filed on Dec. 2, 2021, each of which is hereby incorporated by reference herein in its entirety.

### FIELD OF THE INVENTION

Embodiments of the present invention are directed toward surgical procedures for augmenting weight loss, specifically a method of creating a pylorus-sparing gastric myotomy.

### BACKGROUND

A high percentage of the world population suffers from morbid obesity. In the United States alone the number is estimated to exceed 10 million people. Patients with obesity are susceptible to an increased risk of many medical conditions: heart disease, stroke, diabetes, pulmonary disease, hypertension, gall bladder disease, osteoarthritis, sleep apnea and other breathing problems, and some forms of cancer (e.g., uterine, breast, colorectal, kidney, and gall bladder).

Surgical treatment options are expanding. There are malabsorptive approaches and restrictive approaches. There are malabsorptive approaches as seen with longer limb bypass procedures and restrictive approaches such as the laparoscopic adjustable gastric band and the older vertical banded gastroplasty. Malabsorptive surgical procedures modify the gastrointestinal tract so that only a small fraction of the food and fluid intake is actually digested. Restrictive surgical procedures limit an amount of food and fluid intake. Following a restrictive procedure, a patient's ability to cat is significantly restricted. The patient can only cat a limited amount of food and fluid and any attempt to eat more will result in discomfort. There are also foregut and hindgut mechanisms that rely on alteration in gut hormones and may be an important component to some procedures.

A leading surgical approach is the Roux-en-Y gastric bypass procedure. This procedure combines restrictive mechanisms while bypassing a portion of the stomach and the proximal small intestine. The stomach is restricted by stapling at least a portion of the stomach, creating a pouch and limiting the size of a patient's stomach, thereby restricting a patient's food and fluid intake. Staple line failures are a known problem with this technique. To prevent this, some surgeons make the division more secure by suturing off the pouch from the portion of the stomach that is to remain a part of the digestive tract. The creation of pouches is not generally desirable because they can result in stenosis, e.g., stricture of the stomach stoma that can have a major effect on a patient's eating, and dilation, e.g., stretching of the stomach that can result in weight gain. About 5 to 10 percent of Roux-en-Y patients have dilation problems and about 2 percent have intestinal obstruction. Further, metabolic complications can occur following a Roux-en-Y procedure, for example anemia and calcium deficiency, because essential vitamins and nutrients of blood production (e.g., iron and vitamin B12) depend on the stomach and intestine for absorption, and because calcium is best absorbed in the duodenum, which is bypassed in the procedure. In addition, this procedure is difficult to adjust and impossible to reverse.

Some newer less invasive endoscopic procedures have been developed to target portions of gastric physiology. Some intragastric balloons have been found to delay gastric emptying while they are in place, and cause prolonged fullness with subsequent weight loss. Unfortunately, most endoscopic procedures are only temporary and require device removal after a limited period of time, which is not optimal for a chronic disease such as obesity.

It is thus desirable to provide a new surgical procedure for treating obesity that does not create pouches or remove portions of the stomach, does not use staples, is durable, and can be easily modified.

### SUMMARY

Embodiments of the present invention include methods for augmenting weight loss by a pylorus-sparing gastric myotomy. By performing a pylorus-sparing gastric myotomy, the antral pump phase of gastric digestive physiology is disrupted, and a delay in gastric emptying is induced for the purpose of weight loss.

The procedure of the present invention may be done laparoscopically, endoscopically, perorally, and/or robotically. The medical professional begins by making an incision in a patient for the purpose of creating a submucosal tunnel in the gastric tissue of the gastric antrum and/or gastric body. A submucosal tunnel is created using standard surgical techniques. The submucosal tunnel may extend from the proximal gastric antrum or gastric body to the pre-pyloric area.

After the submucosal tunnel is created, the medical professional performs a pylorus-sparing antral/gastric myotomy in the submucosal tunnel. The pylorus-sparing myotomy may extend from the pre-pyloric area to just distal of the opening of the submucosal tunnel in the gastric antrum or gastric body. Two or more myotomies may be created within the submucosal tunnel. The two or more myotomies may be created in a common tunnel or in adjacent tunnels. The two or more myotomies may be generally parallel to one another, or may not be substantially parallel. The one or more myotomies may be full-thickness, or partial-thickness muscular incisions. In an embodiment, the pylorus remains intact so as to slow gastric emptying.

After the myotomies are formed, the medical professional closes the submucosal tunnel using standard surgical techniques. This may be achieved using sutures with running, interrupted, or other patterns, clips, t-tags, and/or other closure devices.

After the one or more myotomies are created, the medical professional may perform a running suture along the greater curvature and/or posterior wall of the stomach. This serves a division between the myotomies and an optional endoscopic gastroplasty that may be performed.

In some embodiments, the medical professional may perform an endoscopic gastroplasty in the gastric body and/or fundus. The suture pattern of the endoscopic gastroplasty may be alternating U and interrupted sutures. The pattern may also be any combination of U and/or interrupting sutures, or other suturing or plication pattern.

### BRIEF DESCRIPTION OF THE DRAWINGS

FIG. 1 depicts creation of a submucosal tunnel in a patient.

FIG. 2 depicts the submucosal tunnel extending toward the intact pylorus from a gastric lumen perspective.

FIG. 3 depicts a myotomy in the submucosal tunnel stopping short of the pylorus.

FIG. 4 depicts a double myotomy within the submucosal tunnel.

FIG. 5 depicts the access site in the foreground and a normal pylorus in the distance.

FIG. 6 depicts suturing closed the submucosal tunnel.

FIG. 7 depicts a running stitch along the greater curvature and posterior wall of the stomach.

FIG. 8 depicts an endoscopic gastroplasty.

FIG. 9 depicts the appearance of a gastric anatomy post-procedure.

FIG. 10 depicts a normal human gastric anatomy.

FIG. 11 depicts a side illustration of a gastric anatomy post-procedure.

FIGS. 12A-12B depict baseline CO2 excretion rates for patients before the procedures of the present disclosure.

FIGS. 13A-13B depict CO2 excretion rates for patients after the procedures of the present disclosure.

### DETAILED DESCRIPTION

Embodiments of the present invention include methods for augmenting weight loss by a pylorus-sparing gastric myotomy. A medical professional creates a submucosal tunnel in the gastric body of the patient. This may be done laparoscopically, endoscopically, perorally, or robotically. The submucosal tunnel may be approximately 8 cm in length and a horizontal incision. The tunnel may extend from the proximal gastric antrum to the pre-pyloric area. More than one tunnel may be performed as needed.

The medical professional then performs a pylorus-sparing myotomy in the submucosal tunnel, extending from proximal gastric antrum/distal gastric body to the pre-pyloric area, approximately 5 cm long. This may be performed by proceeding in either direction (tunnel-opening to tunnel end, or tunnel-end to tunnel-opening). This may be done longitudinally, diagonally, or horizontally. The pylorus remains intact. The myotomy may be full or partial thickness through the gastric muscle. Alternatively, two or more myotomies may be created in the submucosal tunnel. A myotomy may be created in the antrum and may be extended to the gastric body.

After creation of the myotomy and/or myotomies, the medical professional closes the submucosal tunnel with sutures or clips, using standard techniques.

The medical professional may then perform a running suture along the greater curvature and posterior wall of the stomach. This may serve as a division between a myotomy and an optional endoscopic gastroplasty and to minimize tension on the access site closure that can be created by the endoscopic gastroplasty sutures.

In some embodiments, the medical professional may then perform an endoscopic gastroplasty (endoscopic sleeve gastroplasty or gastric plication procedure) in the gastric body and/or fundus. This may be done with standard techniques as part of this new procedure.

There are many unique benefits to this procedure. The pylorus-sparing gastric myotomy is designed so as to disrupt the antral pump phase of gastric digestive physiology, with the intent of inducing a delay in gastric emptying for the purpose of weight loss. This has the effect of triggering earlier and prolonged fullness during and after a meal. This procedure is unique due to the location of the myotomy and due to the physiologic effects that result from the procedure. An endoscopic myotomy is performed to induce a delay in gastric emptying and a myotomy is being performed for the purposes of inducing weight loss for the treatment of overweight/obesity. This is physiologically possible due to the unique approach and location of the myotomy which leads to a delay in gastric emptying, and limits the effectiveness of the antral pump. This procedure is also unique in that it can be performed together with an endoscopic gastroplasty as part of a unique combination procedure. In this case the two elements are designed to alter gastric physiology in different ways, to augment weight loss of the gastroplasty procedure.

Gastroplasty procedures narrow and shorten the stomach, disrupting transfer of food from the fundus to the antrum and the mixing portion of gastric physiology. Combining a myotomy with a gastroplasty procedure augments weight loss by altering gastric physiology. Together these procedures interrupt multiple phases of gastric digestion and delay gastric emptying. The created running suture is placed at the level of the gastric incisura along the greater curve and posterior stomach to isolate the gastric antrum, protecting the access site to the tunnel from excess tension that could be applied from the gastroplasty procedure.

Normal phases of gastric digestion include accommodation in the gastric fundus, mixing of chyme in the gastric body, and emptying of food from the stomach by the antral pump. This procedure alters the methods of digestion by altering gastric physiology and inducing lower amplitude contractions in the antral pump portion of digestion resulting in a delay in gastric emptying.

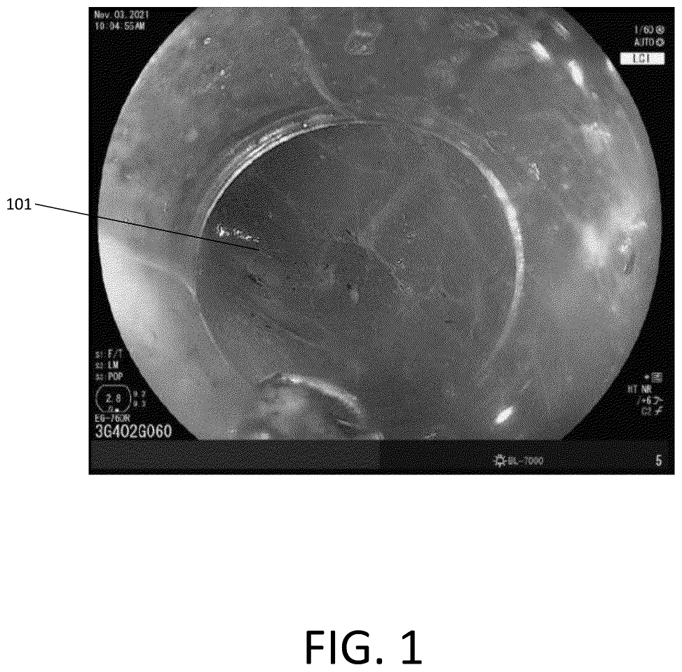

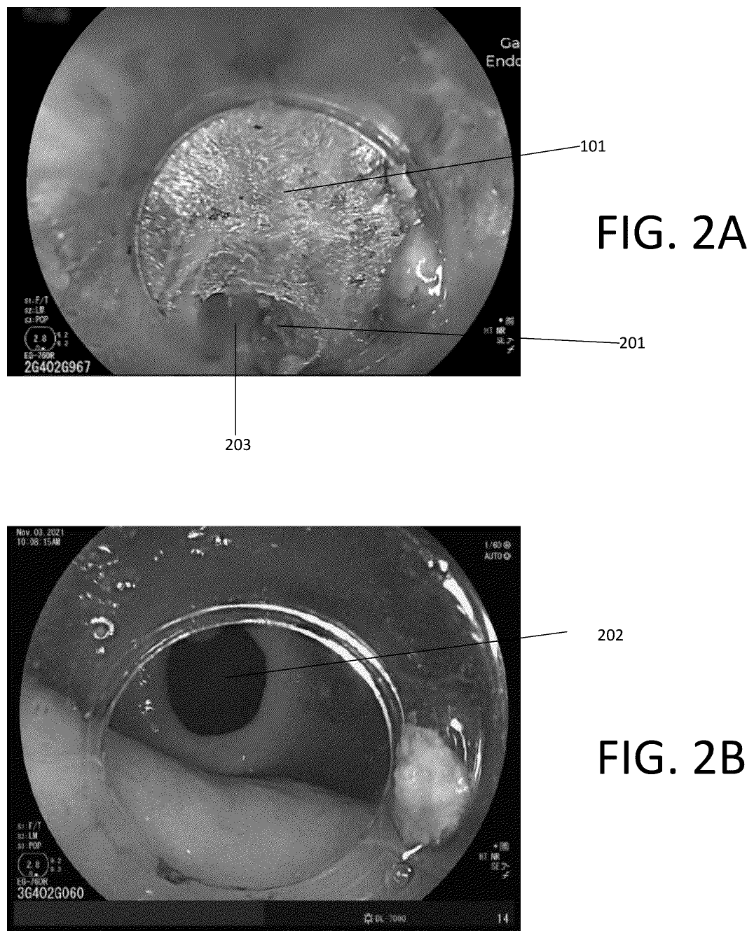

FIGS. 1 and 2A-2B depict formation of a submucosal tunnel. This may be done endoscopically, laparoscopically, perorally, and/or robotically. A horizonal mucosal incision may be made to gain access to the submucosal space 101. A horizontal orientation facilitates subsequent suture closure. FIG. 1 depicts access to the submucosal space 101 after the mucosal incision is made, with an intact submucosal space. After the incision is made, the medical professional proceeds to make a tunnel 201 in the submucosal space 101. Fluid may be injected through an electrosurgical knife 203 as necessary to facilitate creation of the tunnel.

As is shown in FIG. 2A, the medical professional advances the electrosurgical knife 203 through the submucosal area 101. This is done using standard surgical techniques.

FIG. 2B depicts a completed submucosal tunnel 201. The tunnel extends from the proximal gastric antrum 902 to the pre-pyloric area 202. It should be noted that the pylorus 202 remains intact so as to slow gastric emptying. The submucosal tunnel 201 stops short of the pylorus 202, as is shown in FIG. 2B. The tunnel 201 may be approximately 8 cm in length, allowing ample space for one or more myotomies 301.

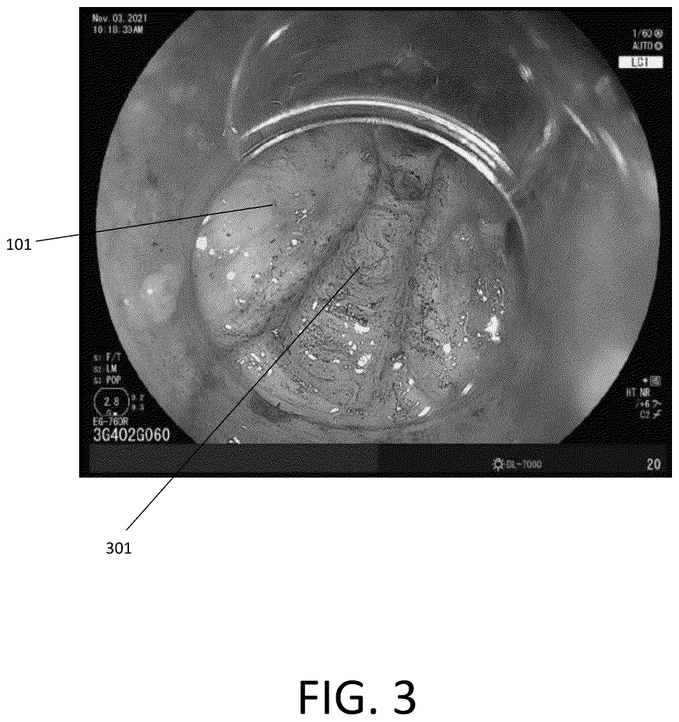

After a submucosal tunnel 201 is created, the medical professional may then create one or more pylorus-sparing myotomies 301 in the submucosal tunnel 201. This may be done endoscopically, laparoscopically, perorally, and/or robotically. The one or more myotomies 301 extend from the pre-pyloric 202 area to just distal of the tunnel opening 601, leaving the pylorus 202 intact. FIG. 3 depicts a partial thickness submucosal myotomy 301 within the submucosal tunnel 201. A created myotomy 301 may also be full thickness. The pylorus 202 is left intact so as to slow gastric emptying. A myotomy 301 may be approximately 5 cm in length. A myotomy 301 may be initiated in the distal antrum 902, and created from distal to proximal, stopping short of the pylorus 202. A myotomy 301 may also be initiated in the pyloric area 202, and created from proximal to distal stopping at the distal antrum 902. The partial thickness myotomy 301 transects circular muscle fibers, leaving longitudinal fibers and the serosa intact. In a full thickness myotomy 301, the medical professional cuts through the serosa.

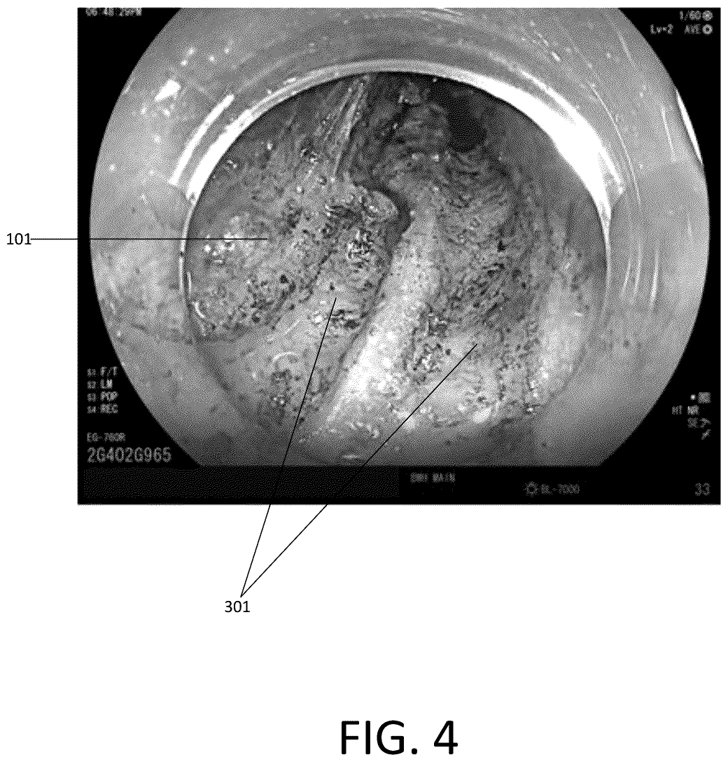

Two or more myotomies 301 may be created in the submucosal area 101. They may be full and/or partial thickness myotomies 301. Two partial thickness myotomies 301 are shown in FIG. 4, though in some embodiments more myotomies 301 may be created. While FIG. 4 shows partial thickness myotomies 301, the two or more myotomies 301 may be full thickness or a combination of full and partial thickness in some embodiments. As is shown in FIG. 4, the two or more myotomies 301 may be substantially parallel to one another. In some embodiments, the two or more myotomies 301 are not substantially parallel to one another.

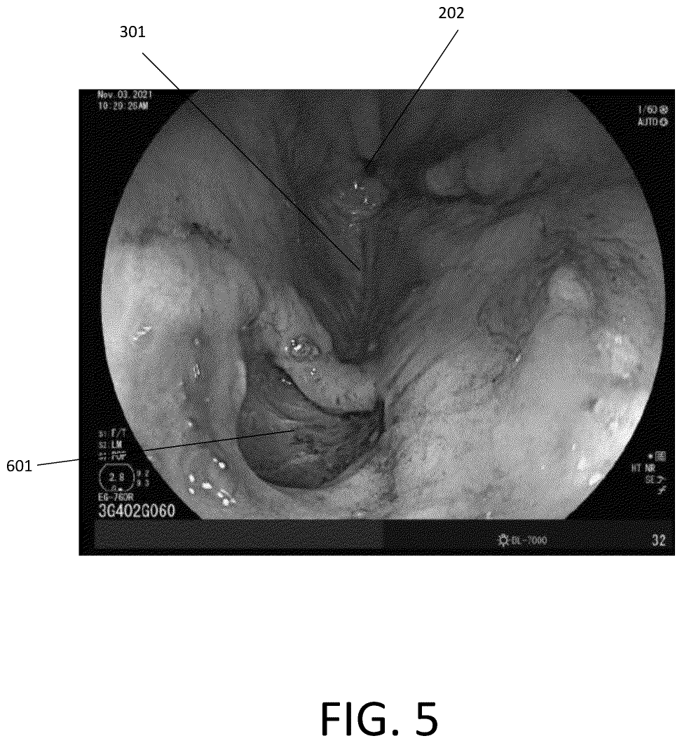

The pylorus 202 remains intact during and after creation of a myotomy 301 in order to slow gastric emptying. As is shown in FIG. 5, the myotomy 301 extends through the submucosal area 101, leaving intact the pylorus 202.

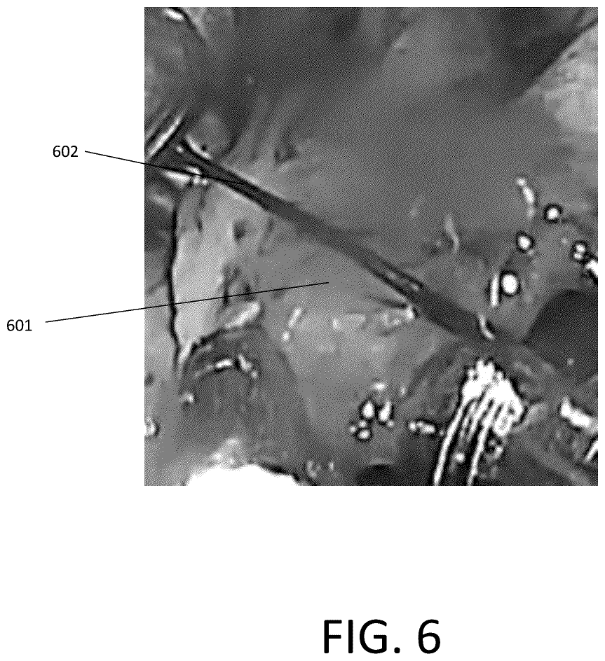

After creation of a myotomy 301, the distal access site 601 to the submucosal layer 101 is closed, as is shown in FIG. 6. The medical professional may close the tunnel opening 601 with sutures and/or clips using standard techniques.

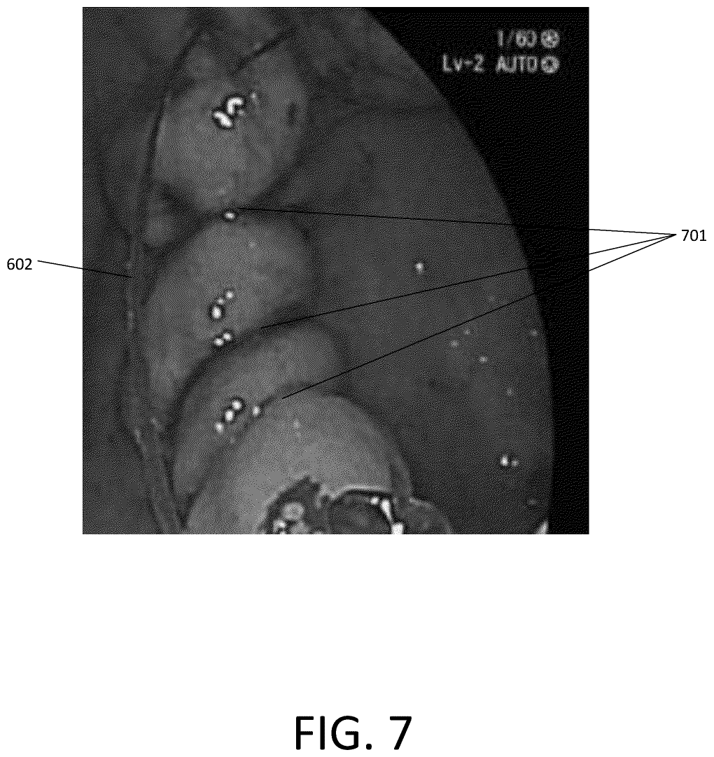

Subsequently, a running suture 701, shown in FIG. 7, separating the gastric body from the antrum 902, is created. The running suture 701 is performed along the greater curvature and posterior wall of the stomach 901. The sutures 701 are placed at the level of the incisura, proximal to the incision site, and is initiated on the anterior surface of the stomach 901. The sutures 701 run along the greater curvature of the stomach 901, and terminate on the posterior side of the stomach 901, short of the lesser curvature.

The running suture 701 serves to create a narrowing that delays the transfer of chyme from the gastric body to the antrum 902 and contributes to delay of gastric emptying. The running suture 701 also reduces the amount of longitudinal tension applied to the access site 601 closure.

The running suture 701 serves as a division between the myotomy 301 and an endoscopic gastroplasty that may be subsequently created, to minimize tension on the access site 601 closure that can be created by endoscopic gastroplasty sutures.

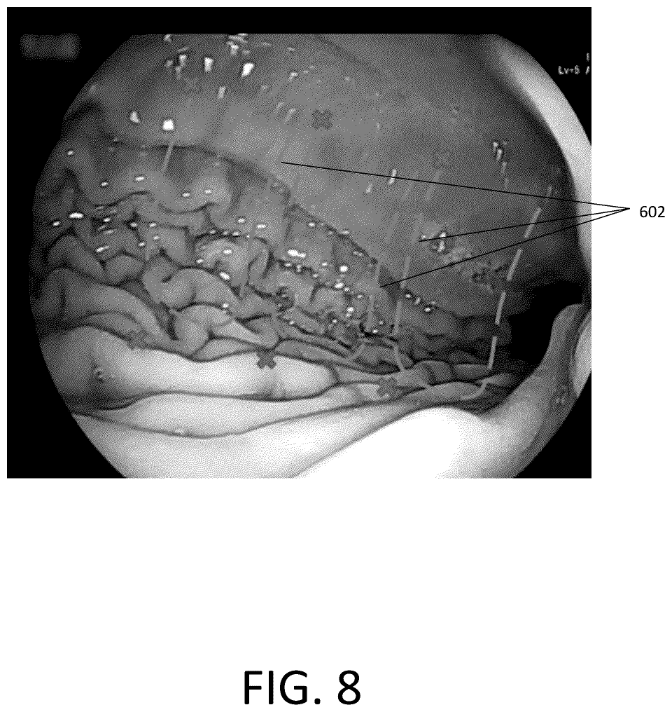

In some embodiments, an endoscopic gastroplasty may be performed. An endoscopic gastroplasty may be performed in the gastric body and/or fundus 903 using a standard technique. As is shown in FIG. 8, an endoscopic gastroplasty may be performed by placing sutures 602 and/or tissue anchors in the gastric body and/or fundus 903. The suture pattern of the endoscopic gastroplasty may be an alternating pattern of U and interrupted sutures 602. The suture pattern may also be any combination of U and/or interrupted sutures 602.

Gastroplasty procedures narrow and shorten the stomach 901, disrupting transfer of food from the fundus 903 to the antrum 902 and the mixing portion of gastric physiology

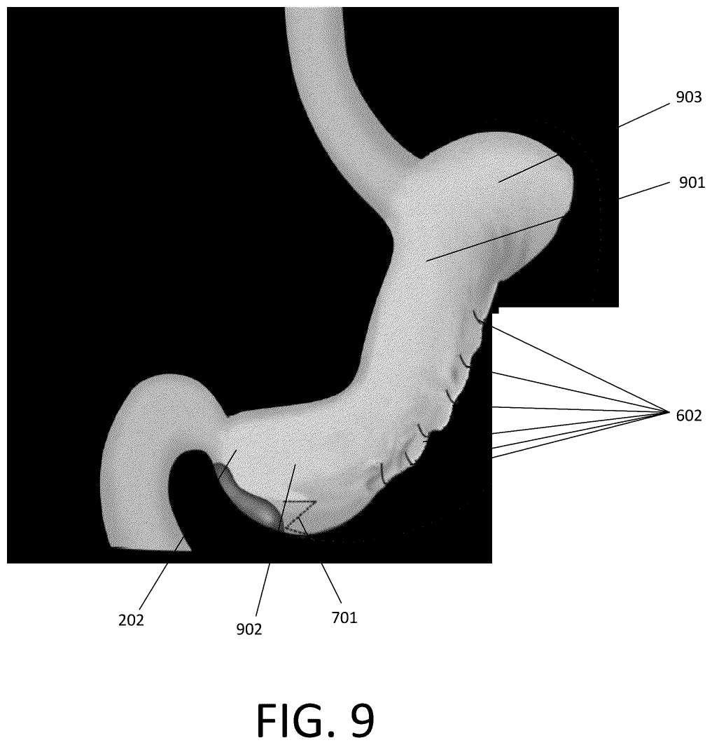

FIG. 9 depicts a final appearance of a patient's stomach 901 following the above-described procedure. An overall smaller stomach 901 size compared to a normal stomach 901 is observed due to a sutured closure in the proximal antrum 902 and sutures in the gastric body consistent with an endoscopic gastroplasty procedure.

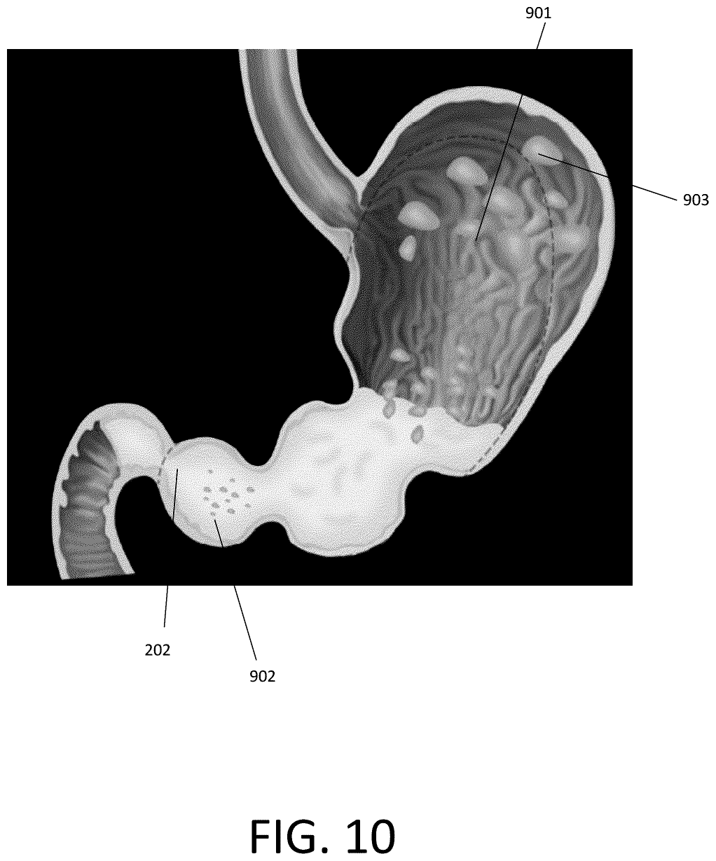

FIG. 10 depicts a normal human stomach 901, showing normal motility and emptying of gastric contents.

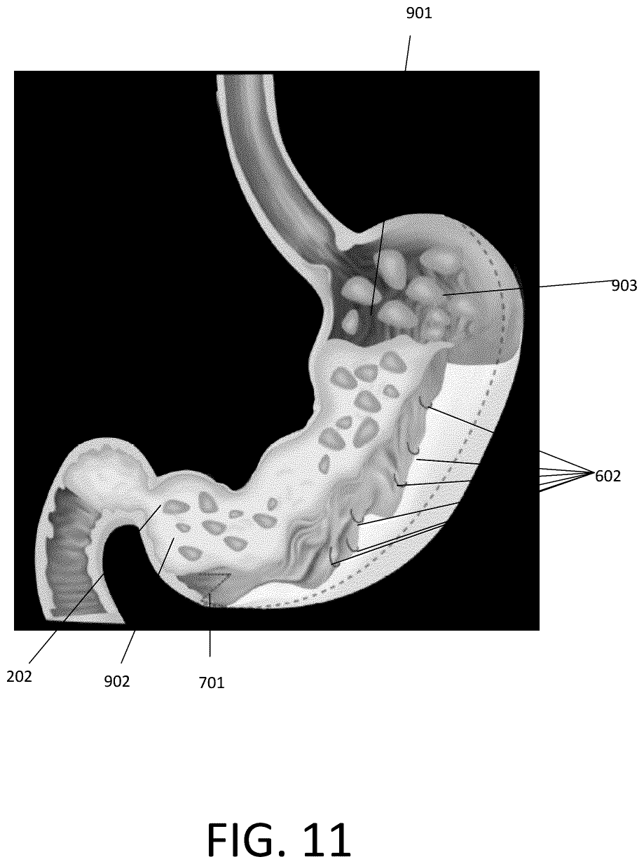

FIG. 11 depicts a human stomach 901 after the above-described procedure. The antral 902 myotomy 301, suture closure 701 of a proximal antrum 902, and sutures 602 in the gastric body consistent with the gastroplasty are shown. The stomach 901 is overall smaller, resulting in lower amplitude contractions in the antral pump portion of digestion and a delay in gastric emptying, leading to augmented weight loss.

### Example 1

A gastric emptying breath test (Cairn Diagnostics, ¹³C-Spirulina Gastric Emptying Breath Test) was performed both before (baseline) and post-procedure.

The results are shown below.

TABLE 1
Pre and Post-Procedure Gastric Emptying Rates

| | Pre- | Post |
|---|---|---|
| | procedure | Procedure |
| | gastric | gastric |
| | emptying | emptying |
| | time via | time via |
| | breath test | breath test |
| Subject | Pre T1/2 | Post T1/2 |
| Number | (min) | (min) |
| Subject | 73.8 | 188.7 |
| 1 | | |
| Subject | 58.2 | 185.3 |
| 2 | | |
| Subject | 97.6 | 216.1 |
| 3 | | |
| Subject | 204.0 | 208.1 |
| 4 | | |
| Subject | 58.1 | 193.3 |
| 5 | | |
| Subject | 49.9 | 230.1 |
| 6 | | |

As shown in the above Table 1, all patients subsequently demonstrated a significant delay in gastric emptying when compared to baseline, as intended by the procedure-confirming the proposed mechanism of action. This is also associated with weight loss, and long-term follow-up is ongoing.

### Example 2

The pylorus-sparing gastric myotomy has also been performed on its own, without gastric plication, in five patients. The tunnel was performed as described above extending from the antral-body junction and extending to the pre-pyloric antrum. The myotomy, or in some cases two roughly parallel myotomies, was performed in the tunnel extending from the pre-pyloric antrum to approximately 2 cm from the mucosal access site at the antral-body junction. The mucosal access site was then closed in standard fashion, using endoscopic sutures or clips. The first patient to complete this protocol experienced a 7.9% TWL at 1 month, going from 158 to 138 pounds. Their gastric emptying T^{1/2} was also markedly delayed on 1-month follow-up going from 56.6 min to 142.7 min (FIGS. 10A and 10B). Additionally, the patient did not experience any symptoms of pathological gastroparesis, with the GCSI score going from 1 at baseline to 7 following the procedure, representing only a change/improvement in the satiety subscale—with earlier and prolonged fullness. The four additional patients have experienced similar weight loss, with an 11.4% TWL at 1-month and 16.8% TWL at 2-months for the entire cohort. They also show improvement in satiety on GCSI, with gastric emptying tests and gut hormone levels pending.

### Example 3

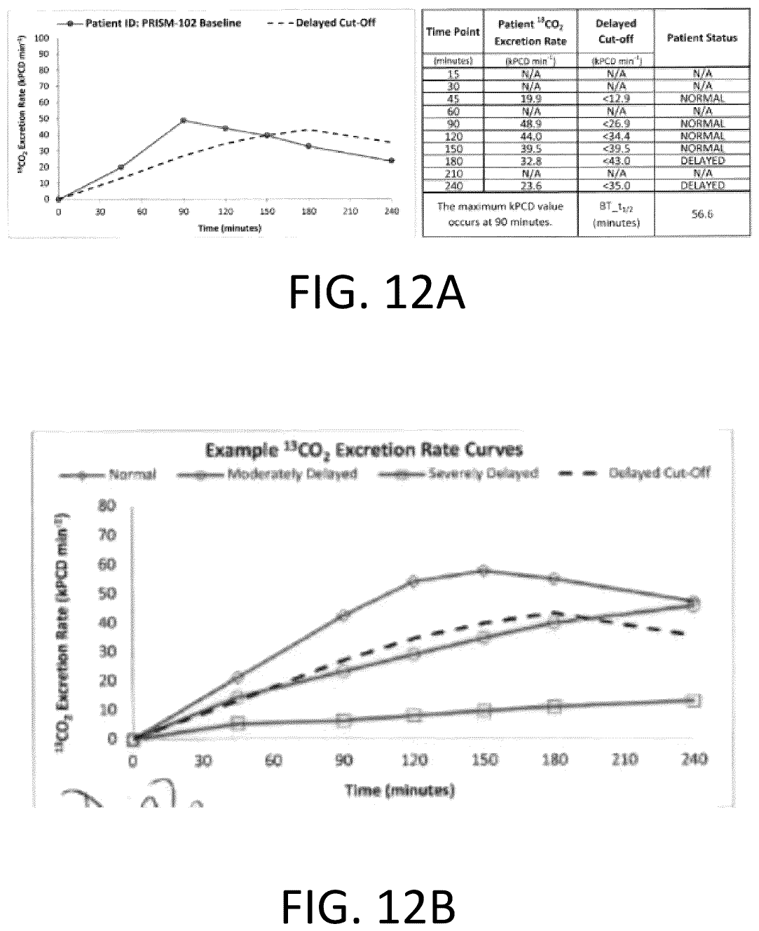

FIG. 12A depicts baseline CO2 excretion rates for patients before the above described procedure. FIG. 12B depicts interpretive guidelines for the data of FIG. 12A.

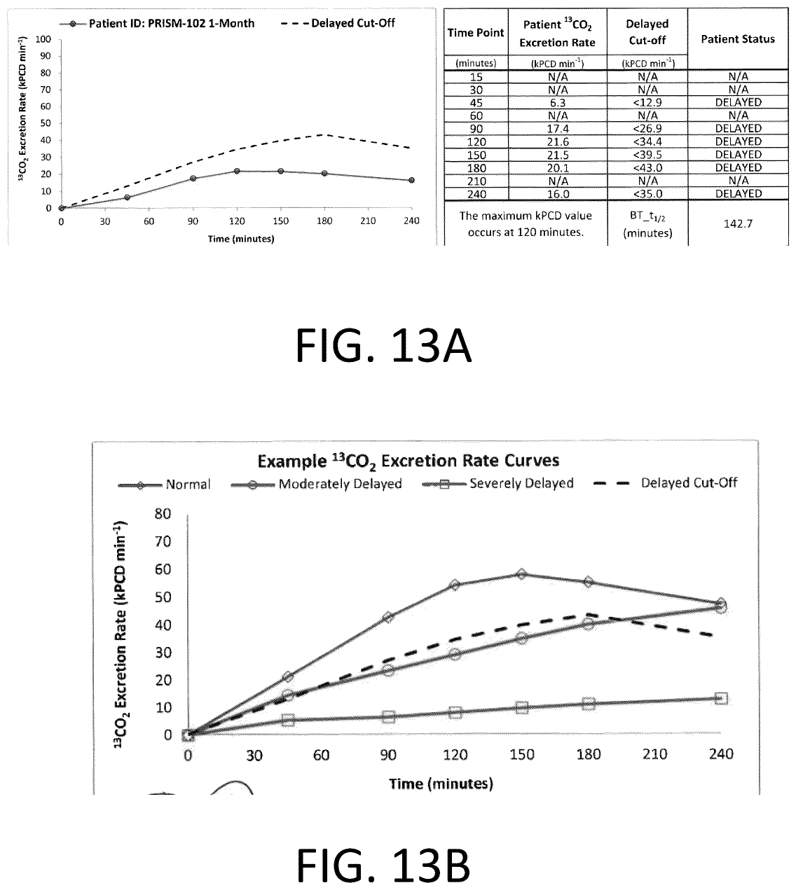

FIG. 13A depicts CO2 excretion rates for patients 1 month after the above described procedure. FIG. 13B depicts interpretive guidelines for the data of FIG. 13A.

Increasing kPCD values (CO2 excretion rates) reflect increasing rates of gastric emptying. Subjects with normal rates of gastric emptying typically display kPCD that exceed cut-off points, reach a maximum between 120-180 minutes and then decline. Subjects with delayed rates of gastric emptying do not decline, they display lower kPCD values, typically (but not always) lower than the cut-off points, rise continuously and reach maximum excretion at 240 minutes. Moderately delayed subjects will have lower kPCD values that appear normal at the 180 and 240 minute time points because their excretion curves are still rising at these times.

What is claimed is:

1. A method of augmenting weight loss in a mammalian patient, the method comprising:
creating a submucosal tunnel in a gastric antrum and/or gastric body of a patient,
wherein the submucosal tunnel extends from a proximal gastric antrum or gastric body of the patient to a pre-pyloric area of the patient;
performing a pylorus-sparing antral myotomy in the submucosal tunnel,
wherein the pylorus-sparing antral myotomy extends from the pre-pyloric area to just distal of an opening of the submucosal tunnel.

2. The method as described in claim 1 further comprising:
performing a running suture along a greater curvature and posterior wall of a stomach of the patient.

3. The method as described in claim 2 further comprising:
performing an endoscopic gastroplasty in the gastric body and/or fundus.

4. The method as described in claim 3 wherein a suture pattern of alternating U and interrupted sutures is used.

5. The method as described in claim 1 wherein two or more myotomies are performed.

6. The method as described in claim 5 wherein at least a portion of the two or more myotomies are generally parallel to another myotomy.

7. The method as described in claim 1 wherein the myotomy is performed endoscopically, laparoscopically, perorally, and/or robotically.

8. The method as described in claim 1 wherein the pylorus-sparing antral myotomy is a full-thickness incision.

9. The method as described in claim 1 wherein the pylorus-sparing antral myotomy is a partial thickness incision.

10. The method as described in claim 1 wherein the pylorus is left intact.

11. A method of augmenting weight loss in a mammalian patient, the method comprising:
creating a submucosal tunnel in a gastric antrum and/or gastric body of a patient,
wherein the submucosal tunnel extends from a proximal gastric antrum or gastric body of the patient to a pre-pyloric area of the patient;
performing a pylorus-sparing antral/gastric myotomy in the submucosal tunnel,
wherein the pylorus-sparing antral myotomy extends from the pre-pyloric area to just distal of an opening of the submucosal tunnel in the gastric antrum or gastric body;
closing the submucosal tunnel;
performing a running suture along a greater curvature and posterior wall of a stomach of the patient.

12. The method as described in claim 11 further comprising:
performing an endoscopic gastroplasty in the gastric body and/or fundus.

13. The method as described in claim 12 wherein a suture pattern of alternating U and interrupted sutures is used, or other suturing or plication pattern.

14. The method as described in claim 11 wherein two or more myotomies are performed a common tunnel and/or in an adjacent tunnel.

15. The method as described in claim 14 wherein at least a portion of the two or more myotomies are generally parallel to the other myotomy.

16. The method as described in claim 11 wherein the myotomy is performed endoscopically, laparoscopically, perorally, and/or robotically.

17. The method as described in claim 11 wherein the pylorus-sparing antral myotomy is a full-thickness muscular incision.

18. The method as described in claim 11 wherein the pylorus-sparing antral myotomy is a partial thickness incision.

19. The method as described in claim 11 wherein the pylorus is left intact.

20. A method of augmenting weight loss in a mammalian patient, the method comprising:
creating a submucosal tunnel in a gastric body of a patient,
wherein the submucosal tunnel extends from a proximal gastric antrum and/or gastric body of the patient to a pre-pyloric area of the patient;
performing a pylorus-sparing antral myotomy in the submucosal tunnel,
wherein the pylorus-sparing antral myotomy extends from the pre-pyloric area to just distal of an opening of the submucosal tunnel;
closing the submucosal tunnel using suturing using a running, interrupted, or other pattern of sutures, clips, t-tags, or other closure devices; and
performing an endoscopic gastroplasty in the gastric body and/or fundus.
